(19) United States
(12) Patent Application Publication
(10) Pub. No.: US 2011/0092850 A1
(43) Pub. Date: Apr. 21, 2011

(54) MRI Biopsy Targeting Guide with Rotational Lock

(76) Inventors: Abhijit G. Kulkarni, Pune (IN); Santosh G. Deshmukh, Pune (IN); Ajay D. Pawar, Pune (IN); Anil R. Jadhav, Pune (IN); Nitin P. Wale, Pune (IN)

(21) Appl. No.: 12/580,321

(22) Filed: Oct. 16, 2009

(51) Int. Cl. A61B 10/02

(52) U.S. Cl. 600/562

(57) ABSTRACT

A biopsy system comprises a control module, a localization assembly, a biopsy device, and a targeting device. A probe and/or other associated components of the biopsy device are configured to selectively couple with the targeting device, which is configured to selectively couple with a grid plate. The targeting device may comprise a rotational lock for securing the targeting device within the grid plate. The targeting device may further comprise an elastomeric insert positioned within guide holes of the targeting device for securing the probe and/or other associated components within the guide hole of the targeting device. The guide holes of the targeting device may alternatively, or in addition, further comprise one or more retaining rings positioned within guide holes of the targeting device for securing the probe and/or other associated components within the guide hole of the targeting device.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. Merely exemplary biopsy devices are disclosed in U.S. Pat. No. 6,273,862, entitled “Surgical Device for the Collection of Soft Tissue,” issued Aug. 14, 2001; U.S. Pat. No. 6,231,522, entitled “Biopsy Instrument with Breakable Sample Segments,” issued May 15, 2001; U.S. Pat. No. 6,228,055, entitled “Devices for Marking and Defining Particular Locations in Body Tissue,” issued May 8, 2001; U.S. Pat. No. 6,120,462, entitled “Control Method for an Automated Surgical Biopsy Device,” issued Sep. 19, 2000; U.S. Pat. No. 6,086,544, entitled “Control Apparatus for an Automated Surgical Biopsy Device,” issued Jul. 11, 2000; U.S. Pat. No. 6,077,230, entitled “Biopsy Instrument with Removable Extractor,” issued Jun. 20, 2000; U.S. Pat. No. 6,017,316, entitled “Vacuum Control System and Method for Automated Biopsy Device,” issued Jan. 25, 2000; U.S. Pat. No. 6,007,497, entitled “Surgical Biopsy Device,” issued Dec. 28, 1999; U.S. Pat. No. 5,980,469, entitled “Method and Apparatus for Automated Biopsy and Collection of Soft Tissue,” issued Nov. 9, 1999; U.S. Pat. No. 5,964,716, entitled “Method of Use for a Multi-Port Biopsy Instrument,” issued Oct. 12, 1999; U.S. Pat. No. 5,928,164, entitled “Apparatus for Automated Biopsy and Collection of Soft Tissue,” issued Jul. 27, 1999; U.S. Pat. No. 5,775,333, entitled “Apparatus for Automated Biopsy and Collection of Soft Tissue,” issued Jul. 7, 1998; U.S. Pat. No. 5,769,086, entitled “Control System and Method for Automated Biopsy Device,” issued Jun. 23, 1998; U.S. Pat. No. 5,649,547, entitled “Methods and Devices for Automated Biopsy and Collection of Soft Tissue,” issued Jul. 22, 1997; U.S. Pat. No. 5,526,822, entitled “Method and Apparatus for Automated Biopsy and Collection of Soft Tissue,” issued Jun. 18, 1996; U.S. Pub. No. 2008/0214955, entitled “Presentation of Biopsy Sample by Biopsy Device,” published Sep. 4, 2008; U.S. Pub. No. 2007/0255168, entitled “Grid and Rotatable Cube Guide Localization Fixture for Biopsy Device,” published Nov. 1, 2007; U.S. Pub. No. 2007/0118048, entitled “Remote Thumbwheel for a Surgical Biopsy Device,” published May 24, 2007; U.S. Pub. No. 2005/0283069, entitled “MRI Biopsy Device Localization Fixture,” published Dec. 22, 2005; U.S. Pub. No. 2003/0199753, entitled “MRI Compatible Biopsy Device with Detachable Probe,” published Oct. 23, 2003; U.S. Pub. No. 2003/0109803, entitled “MRI Compatible Surgical Biopsy Device,” published Jun. 12, 2003; U.S. Pub. No. 2008/0221480, entitled “Biopsy Sample Storage,” published Sep. 11, 2008; and U.S. Pub. No. 2008/0146962, entitled “Biopsy System with Vacuum Control Module,” published Jun. 19, 2008. The disclosure of each of the above-cited U.S. patents and U.S. patent application Publications is incorporated by reference herein.

Some biopsy systems may provide an apparatus to guide a probe and/or other components of a biopsy device to a desired biopsy site. In some such biopsy systems, a guide cube and positioning grid plate may be used. The guide cube may be selectively located within an opening in the grid plate. The guide cube may include guide holes to receive a portion of the probe and/or other components, for example a needle, cannula, obturator, or combinations of these or other components. With the guide cube inserted in the grid plate, the probe or other components can be guided through a selected guide hole of the guide cube to arrive at a desired biopsy site. The desired biopsy site may or may not have been identified and/or targeted by one or more of the guidance approaches mentioned above. In some situations, it might be desirable to provide a guide cube with features that improve a guide cube's use with one or more positioning grid plates. Merely exemplary biopsy device guides are disclosed in U.S. patent application Ser. No. 12/485,119, entitled “Biopsy Targeting Cube with Elastomeric Edges,” filed Jun. 16, 2009; U.S. patent application Ser. No. 12/485,138, entitled “Biopsy Targeting Cube with Elastomeric Body,” filed Jun. 16, 2009; U.S. patent application Ser. No. 12/485,168, entitled “Biopsy Targeting Cube with Malleable Members,” filed Jun. 16, 2009; U.S. patent application Ser. No. 12/485,278, entitled “Biopsy Targeting Cube with Angled Interface,” filed Jun. 16, 2009; and U.S. patent application Ser. No. 12/485,318, entitled “Biopsy Targeting Cube with Living Hinges,” filed Jun. 16, 2009. The disclosure of each of the above-cited U.S. patent applications is incorporated by reference herein.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 is a perspective view of a biopsy system including a control module remotely coupled to a biopsy device, and including a localization assembly.

FIG. 2 is a perspective view of a breast coil of the localization assembly of FIG. 1.

FIG. 3 is a perspective view of the biopsy device inserted through the guide cube of the localization assembly of FIG. 1.

FIG. 4 is a perspective view of the obturator and cannula of the biopsy system of FIG. 1.

FIG. 5 is an exploded perspective view of the obturator and cannula of FIG. 4.

FIG. 6 is a perspective view of the guide cube inserted into the grid plate of the localization assembly of FIG. 1.

FIG. 7 is a perspective view of the obturator and cannula of FIG. 4 with a depth stop device of FIG. 1 inserted through the guide cube and grid plate of FIG. 6.

FIG. 8 is a perspective view of the guide cube of the biopsy system of FIG. 1.

FIG. 9 is a diagram of nine guide positions achievable by rotating the guide cube of FIG. 8.

FIG. 10 is a perspective view of another guide cube for the biopsy system of FIG. 1 with a self-grounding feature.

FIG. 11 is a perspective view of the obturator and cannula of FIG. 1 inserted into one of two guide cubes of FIG. 10 inserted into the grid plate of FIG. 1.

FIG. 12 is a perspective view of another guide cube having an open top and bottom with another self-grounding feature.

FIG. 13 is a rear perspective view of another guide cube with another self-grounding feature.

FIG. 14 is a front perspective view of the guide cube of FIG. 13.

FIG. 15 is a right side view of the guide cube of FIG. 13 with angled, parallel guide holes depicted in phantom.

FIG. 16 is a front perspective view of an exemplary cylindrical guide device having a rear locking feature and o-rings positioned within the guide holes.

FIG. 17 is a front perspective view of another exemplary cylindrical guide device having a rear locking feature and inserts positioned within the guide holes.

FIG. 18 is a front view the cylindrical guide device of FIG. 16 shown in a locked position with a portion of the grid plate of FIG. 6.

FIG. 19 is a front view of the cylindrical guide device of FIG. 16 shown in an unlocked position with a portion of the grid plate of FIG. 6.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

As shown in the figures, an exemplary magnetic resonance imaging (MRI or MR imaging) compatible biopsy system may include a control module (12), localization assembly (15), and biopsy device (14). In particular, localization assembly (15) is configured to localize a patient's breast and guide needle (90) of biopsy device (14) to a targeted area within the patient's breast; while control module (12) is operable to control biopsy device (14) after needle (90) has been introduced to the target site. These components and their sub-components will be discussed further below. In addition, guide devices, including guide cubes, for use with various localization assemblies will be discussed. While this disclosure may reference the biopsy system as compatible with MRI and MRI equipment and devices, it should be appreciated that other imaging techniques and equipment and devices may be used with the components described below, including but not limited to stereotactic, ultrasound, PEM, BSGI, and/or other imaging techniques and equipment.

### I. Control Module

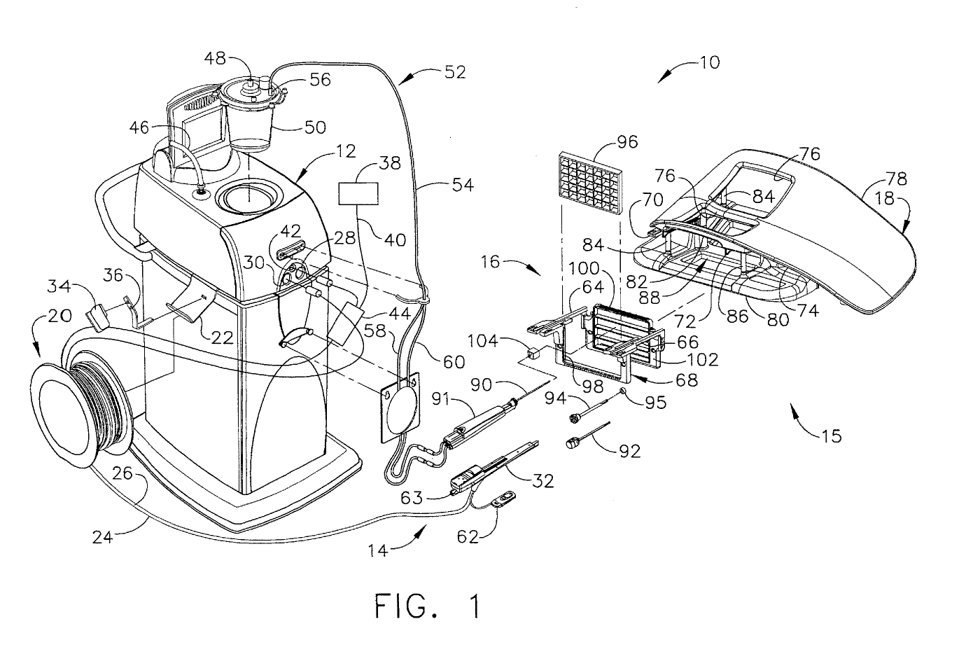

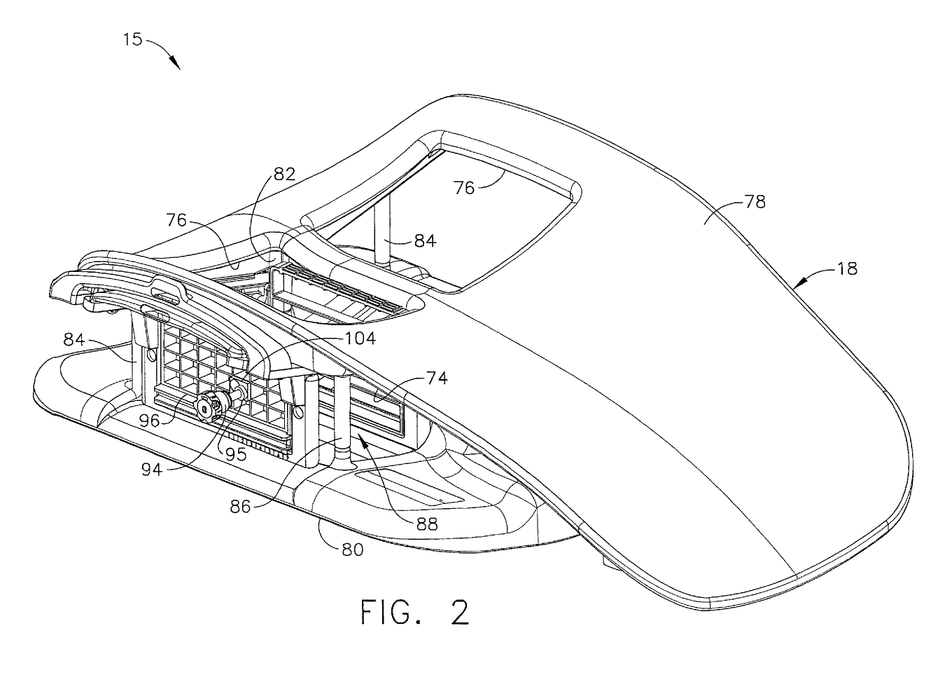

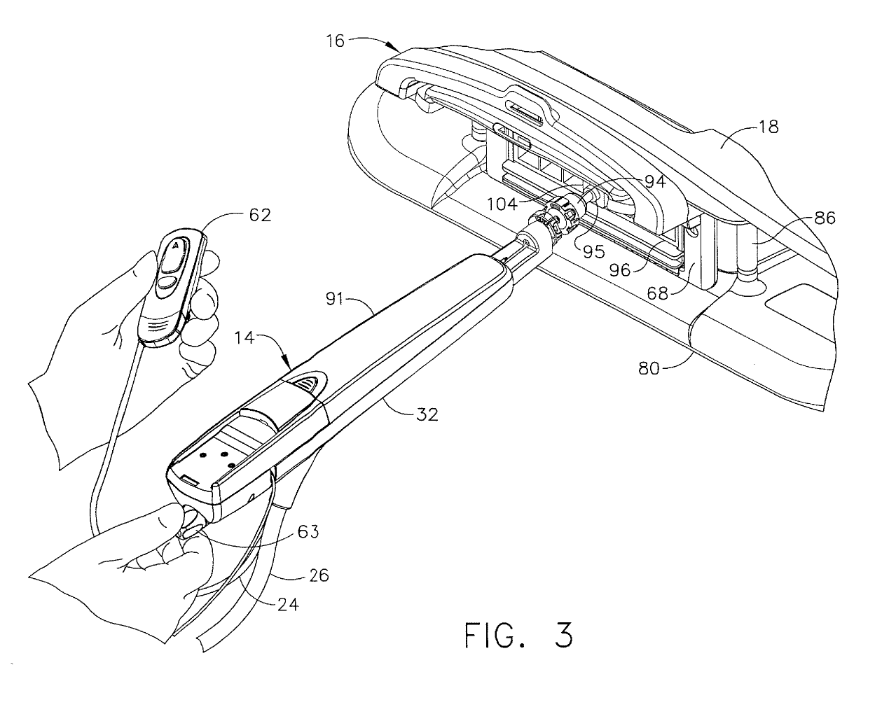

In FIGS. 1-3, MRI compatible biopsy system (10) has control module (12) that may be placed outside of a shielded room containing an MRI machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MM machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MM machine or other machine. In-line enclosure (44) may register tether (40), electrical cable (24) and mechanical cable (26) to their respective ports (42, 28, 30) on control module (12).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pub. No. 2008/0228103, entitled “Vacuum Timing Algorithm for Biopsy Device,” published Sep. 18, 2008, the disclosure of which is incorporated by reference herein. As another merely illustrative example, control module (12) may instead be configured and operable in accordance with the teachings of U.S. patent application Ser. No. 12/337,814, entitled “Control Module Interface for MRI Biopsy Device,” filed Dec. 18, 2008, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

### II. Localization Assembly

Localization assembly (15) of the present example comprises breast coil (18) and localization fixture (16). These components of localization assembly (15) are described further below.

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

### III. Biopsy Device

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

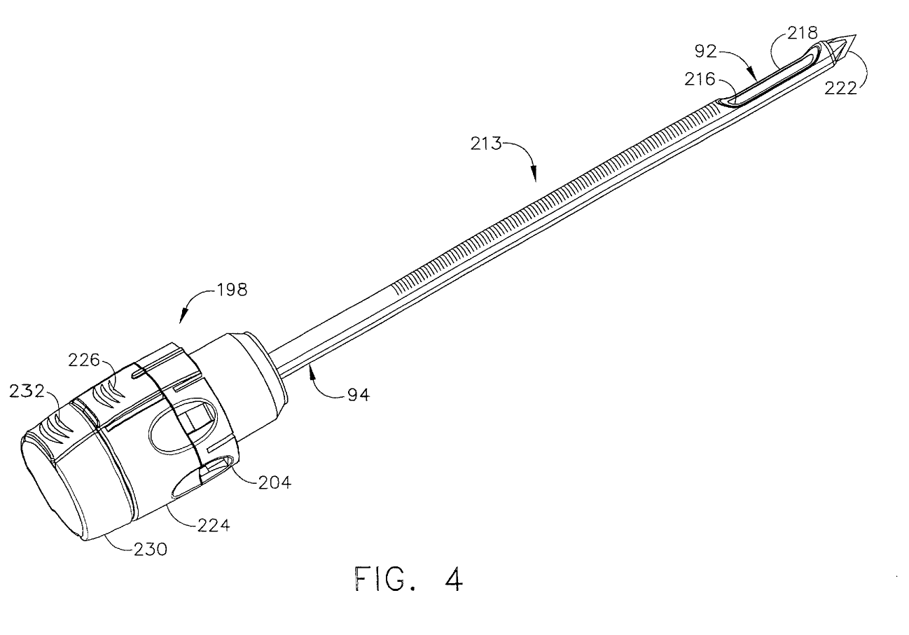

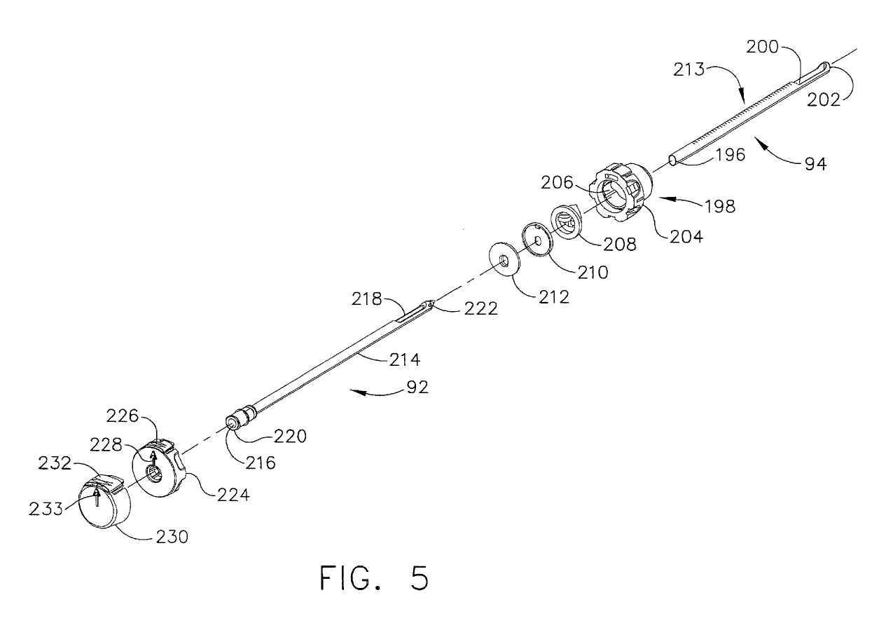

In the present example, cannula (94) and obturator (92) are associated with probe (91). In particular, and as shown in FIGS. 4, 5, and 7, obturator (92) is slid into cannula (94) and the combination is guided through guide cube (104) to the biopsy site within the breast tissue. Obturator (92) is then withdrawn from cannula (94), then needle (90) of probe (91) is inserted in cannula (94), and then biopsy device (14) is operated to acquire one or more tissue samples from the breast via needle (90).

Cannula (94) of the present example is proximally attached to cylindrical hub (198) and cannula (94) includes lumen (196) and lateral aperture (200) proximate to open distal end (202). Cylindrical hub (198) has exteriorly presented thumbwheel (204) for rotating lateral aperture (200). Cylindrical hub (198) has interior recess (206) that encompasses duckbill seal (208), wiper seal (210) and seal retainer (212) to provide a fluid seal when lumen (196) is empty and for sealing to inserted obturator (92). Longitudinally spaced measurement indicia (213) along an outer surface of cannula (94) visually, and perhaps physically, provide a means to locate depth stop device (95) of FIG. 1.

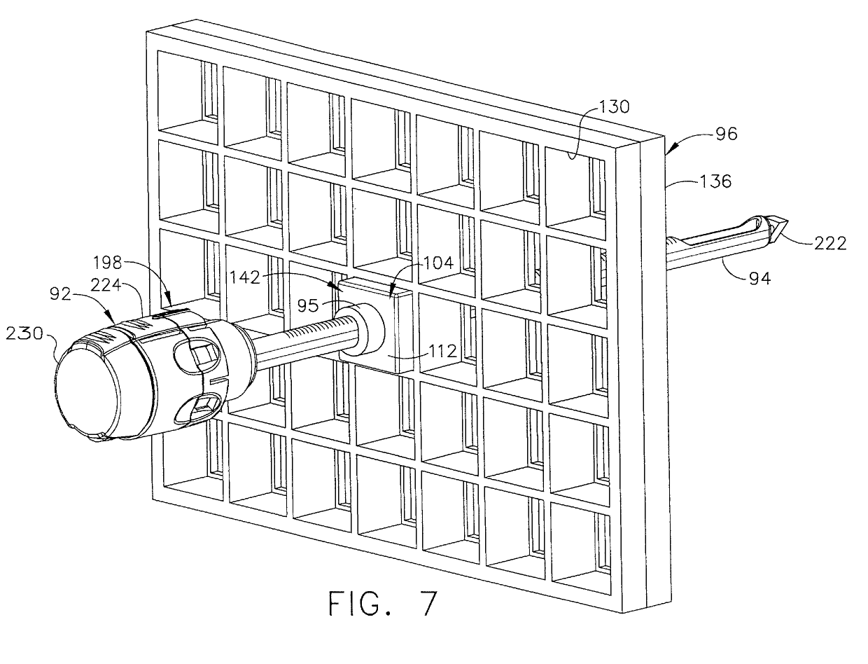

Obturator (92) of the present example incorporates a number of components with corresponding features. Hollow shaft (214) includes fluid lumen (216) that communicates between imageable side notch (218) and proximal port (220). Hollow shaft (214) is longitudinally sized to extend, when fully engaged with cannula (94), piercing tip (222) out of distal end (202) of cannula (94). Obturator thumbwheel cap (224) encompasses proximal port (220) and includes locking feature (226), which includes visible angle indicator (228), that engages cannula thumbwheel (204) to ensure that imageable side notch (218) is registered to lateral aperture (200) in cannula (94). Obturator seal cap (230) may be engaged proximally into obturator thumbwheel cap (224) to close fluid lumen (216). Obturator seal cap (230) of the present example includes locking or locating feature (232) that includes visible angle indicator (233) that corresponds with visible angle indicator (228) on obturator thumbwheel cap (224), which may be fashioned from either a rigid, soft, or elastomeric material. In FIG. 7, guide cube (104) has guided obturator (92) and cannula (94) through grid plate (96).

While obturator (92) of the present example is hollow, it should be understood that obturator (92) may alternatively have a substantially solid interior, such that obturator (92) does not define an interior lumen. In addition, obturator (92) may lack side notch (218) in some versions. Other suitable components, features, configurations, functionalities, operability, etc. for an obturator (92) will be apparent to those of ordinary skill in the art in view of the teachings herein. Likewise, cannula (94) may be varied in a number of ways. For instance, in some other versions, cannula (94) has a closed distal end (202). As another merely illustrative example, cannula (94) may have a closed piercing tip (222) instead of obturator (92) having piercing tip (222). In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop (95). Depth stop may be of any suitable configuration that is operable to prevent cannula (94) and obturator (92) (or needle (90)) from being inserted further than desired. For instance, depth stop (95) may be positioned on the exterior of cannula (94) (or needle (90)), and may be configured to restrict the extent to which cannula (94) is inserted into a guide cube. It should be understood that such restriction by depth stop (95) may further provide a limit on the depth to which the combination of cannula (94) and obturator (92) or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stops (95) that may be used with biopsy system (10) are described in U.S. Pub. No. 2007/0255168, entitled “Grid and Rotatable Cube Guide Localization Fixture for Biopsy Device,” published Nov. 1, 2007, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with lateral aperture (200) of cannula (94) when needle (90) is inserted into lumen (196) of cannula (94). Probe (91) of the present example further comprises a rotating and translating cutter (not shown), which is driven by components in holster (32), and which is operable to sever tissue protruding through lateral aperture (200) of cannula (94) and the lateral aperture of needle (90). Severed tissue samples may be retrieved from biopsy device (14) in any suitable fashion.

By way of example only, biopsy device (14) may be configured and operable in accordance with the teachings of U.S. Pub. No. 2008/0228103, entitled “Vacuum Timing Algorithm For Biopsy Device,” published Sep. 18, 2008, the disclosure of which is incorporated by reference herein. As another merely illustrative example, biopsy device (14) may be configured and operable in accordance with the teachings of U.S. patent application Ser. No. 12/337,874, entitled “Mechanical Tissue Sample Holder Indexing Device,” filed Dec. 18, 2008, the disclosure of which is incorporated by reference herein. As another merely illustrative example, biopsy device (14) may be configured and operable in accordance with the teachings of U.S. patent application Ser. No. 12/337,674, entitled “Biopsy Device with Sliding Cutter Cover,” filed Dec. 18, 2008, the disclosure of which is incorporated by reference herein. By way of example only, cannula (94) may be replaced with any of the detachable needles described in U.S. patent application Ser. No. 12/337,674, entitled “Biopsy Device with Sliding Cutter Cover.” As another merely illustrative example, biopsy device (14) may be configured and operable in accordance with the teachings of U.S. patent application Ser. No. 12/337,911, entitled “Biopsy Device with Discrete Tissue Chambers,” filed Dec. 18, 2008, the disclosure of which is incorporated by reference herein. As another merely illustrative example, biopsy device (14) may be configured and operable in accordance with the teachings of U.S. patent application Ser. No. 12/337,942, entitled “Biopsy Device with Central Thumbwheel,” filed Dec. 18, 2008, the disclosure of which is incorporated by reference herein. Alternatively, biopsy device (14) may have any other suitable components, features, configurations, functionalities, operability, etc. Other suitable variations of biopsy device (14) and associated components will be apparent to those of ordinary skill in the art in view of the teachings herein

### IV. Guide Devices

Guide devices, including guide cubes, described below are generally adapted for use with a localization assembly (15) as described above. Numerous features of merely exemplary guide devices will be discussed in the paragraphs that follow.

### A. Guide Cubes

In some versions a guide device may comprise a guide cube. Guide cubes may comprise a body defined by one or more edges and faces. The body may include one or more guide holes or other types of passages that extend between faces of the guide cube and that may be used to guide an instrument such as a biopsy device (14) or a portion of a biopsy device (14) (e.g., needle (90) of biopsy device (14), a combination of cannula (94) and obturator (92), etc.). Guide cubes may be rotatable about one, two, or three axes to position the one or more guide holes or passages of the guide cube into a desired position.

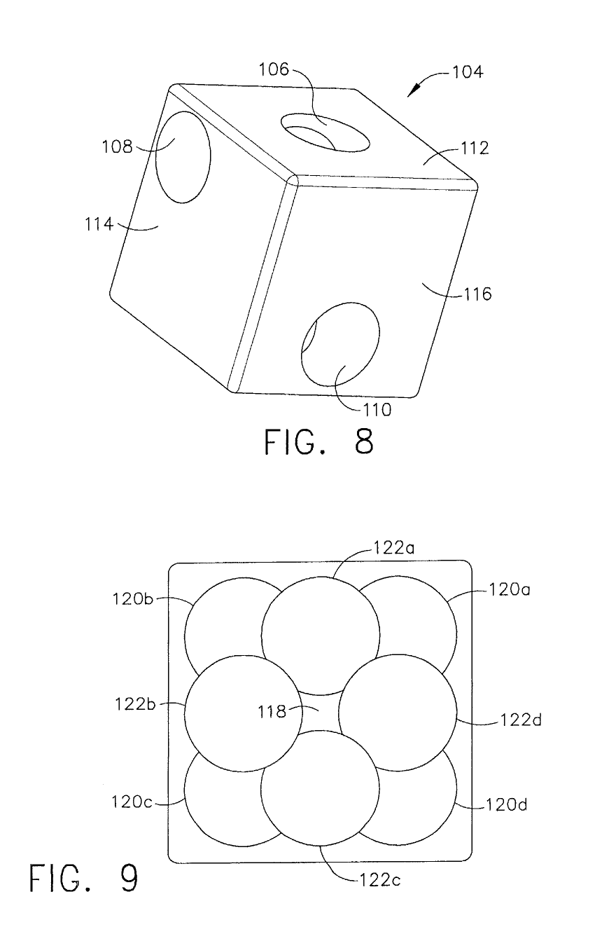

Referring now to FIG. 8, guide cube (104), includes central guide hole (106), corner guide hole (108), and off-center guide hole (110) that pass orthogonally to one another between respective opposite pairs of faces (112, 114, 116). By selectively rotating guide cube (104) in two axes, one pair of faces (112, 114, 116) may be proximally aligned to an unturned position and then the selected proximal face (112, 114, 116) optionally rotated a quarter turn, half turn, or three-quarter turn. Thereby, one of nine guide positions (118, 120a-120d, 122a-122d) may be proximally exposed as depicted in FIG. 9. More specifically, central guide hole (106) may provide for guide position (118), corner guide hole (108) may provide for guide positions (120a-120d), and off-center guide hole (110) may provide for guide positions (122a-122d).

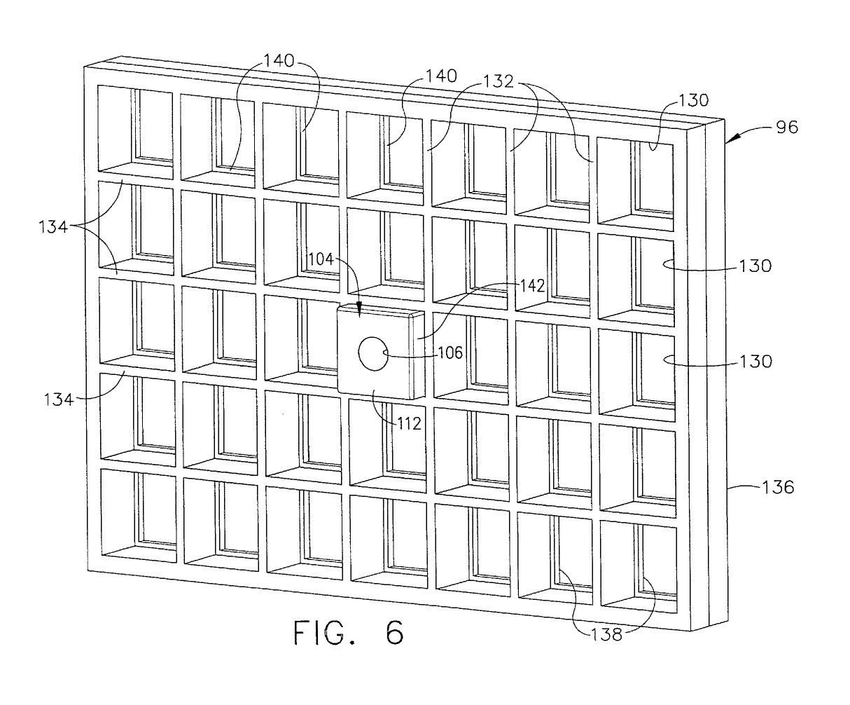

In FIG. 6, two-axis rotatable guide cube (104) is sized for insertion from a proximal side into one of a plurality of square recesses (130) in grid plate (96), which are formed by intersecting vertical bars (132) and horizontal bars (134). Guide cube (104) is prevented from passing through grid plate (96) by backing substrate (136) attached to a front face of grid plate (96). Backing substrate (136) includes respective square opening (138) centered within each square recess (130), forming lip (140) sufficient to capture the front face of guide cube (104), but not so large as to obstruct guide holes (104, 106, 108). The depth of square recesses (130) is less than guide cube (104), thereby exposing a proximal portion (142) of guide cube (104) for seizing and extraction from grid plate (96). It will be appreciated by those of ordinary skill in the art based on the teachings herein that backing substrate (136) of grid plate (96) may be omitted altogether in some versions. In some such versions without backing substrate (136) other features of a guide cube, as will be discussed in more detail below, may be used to securely and removably fit a guide cube within a grid plate. However, such other features may also be used in combination with a grid plate having backing substrate (136), such as grid plate (96), instead of partially or wholly omitting backing substrate (136).

### B. Self-Grounding Guide Cubes

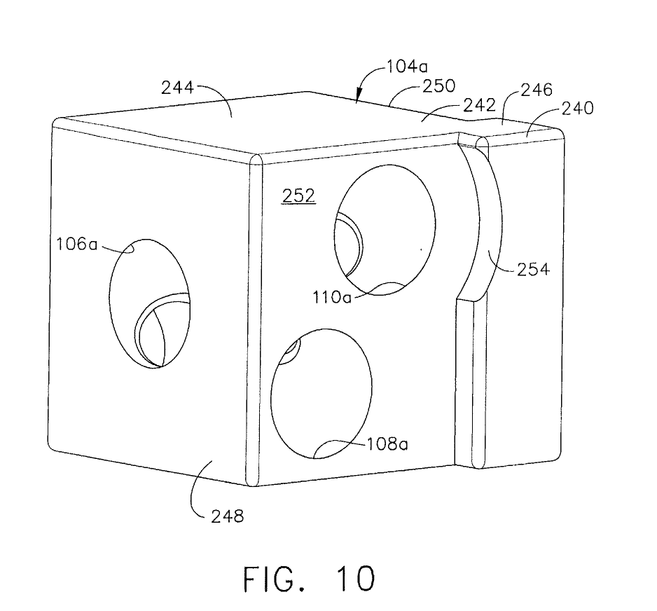

In FIG. 10, guide cube (104a) has self-grounding by means of added rectangular prism (240) which has a shared edge with cubic portion (242) of guide cube (104a). When viewed orthogonally to the shared cube edge, larger square face (244) of cubic portion (242) overlaps with smaller square face (246) of rectangular prism (240). As shown in FIG. 11, rectangular prism (240) allows proximal exposure of one of two adjacent faces (250, 252) of guide cube (104a) and then turning each to one of four quarter-turn rotational positions. In the illustrative version, first face (250) has central guide hole (106a) and second face (252) has corner guide hole (108a), and off-center guide hole (110a). Radial recess (254) is formed in rectangular prism (240) to allow grounding of depth stop device (95) against face (252) when off-center guide hole (110a) is used.

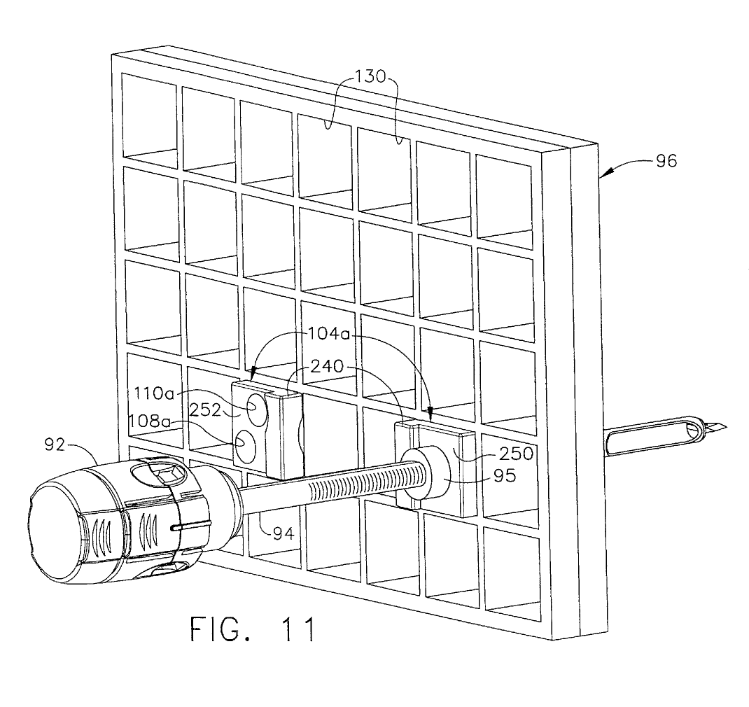

In FIG. 12, guide cube (104b) has self-grounding by means of added rectangular prism (260) that protrudes from two faces (262, 264) of guide cube (104b). Rectangular prism (260) allows proximal exposure of one of two adjacent faces (262, 264) of guide cube (104b) and then turning each to one of four quarter-turn rotational positions. In the illustrative version, first face (262) has central guide hole (106b) and second face (264) has corner guide hole (108b) and off-center guide hole (110b). First radial recess (266) is formed in rectangular prism (260) to allow grounding of depth stop device (95) against face (264) when off-center guide hole (110b) is used. Second radial recess (268) is formed in rectangular prism (260) to allow grounding of depth stop device (95) against face (262) when central guide hole (106b) is used. As discussed in greater detail below, guide cube (104b) may have open top (261) and/or an open bottom (not shown) defined by the faces of guide cube (104b) as depicted in the illustrated version.

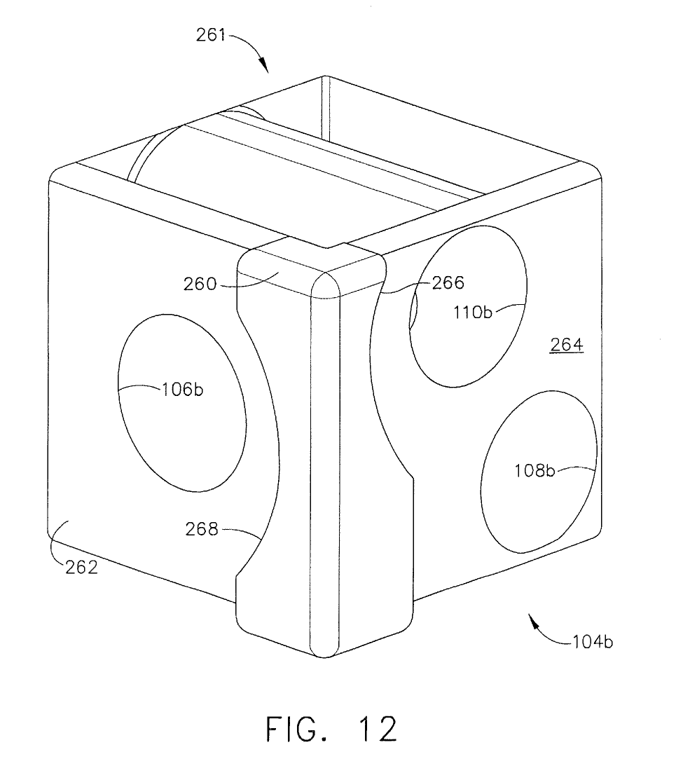

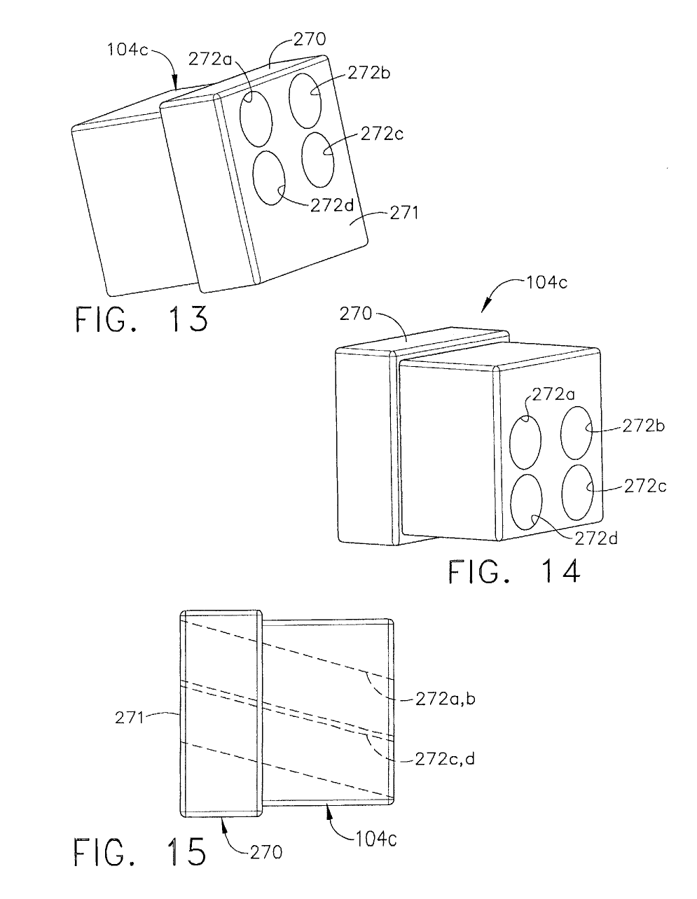

In FIGS. 13-15, guide cube (104c) has proximal enlarged hat portion (270) about proximal face (271) that grounds against selected square recess (130), such as in grid plate (96), and allows rotation about one axis to one of four quarter-turn positions. Four angled guide holes (272a, 272b, 272c, 272d) allow accessing not only an increased number of insertion points within selected square recess (130) but also a desired angle of penetration rather than being constrained to a perpendicular insertion. It will be appreciated based on the teachings herein that while angled guide holes may be used in some versions, orthogonal guide holes may be used instead of or in addition to angled guide holes in other versions.

C. Cylindrical Guide Devices with Rotational Locks

In some versions of guide devices, the guide device may include features that assist in securing the guide device within an aperture of a grid plate. Such features may be designed to secure the guide device from movement in a proximal direction, distal direction, lateral direction, or combinations of these or other directions. In some versions of guide devices, the guide devices may further include features that assist in securing an instrument, such as a biopsy device (14) or a portion of a biopsy device (14) (e.g., needle (90) of biopsy device (14), a combination of cannula (94) and obturator (92), etc.), within a selected guide hole or passageway of the guide device. In some versions, such features may substantially retain the instrument or portion of the instrument by providing resistance to movement of the instrument in a proximal direction, distal direction, lateral direction, or combinations of these or other directions. The paragraphs that follow will describe merely exemplary versions of guide devices or modifications to guide devices that may include some of these optional features, among features.

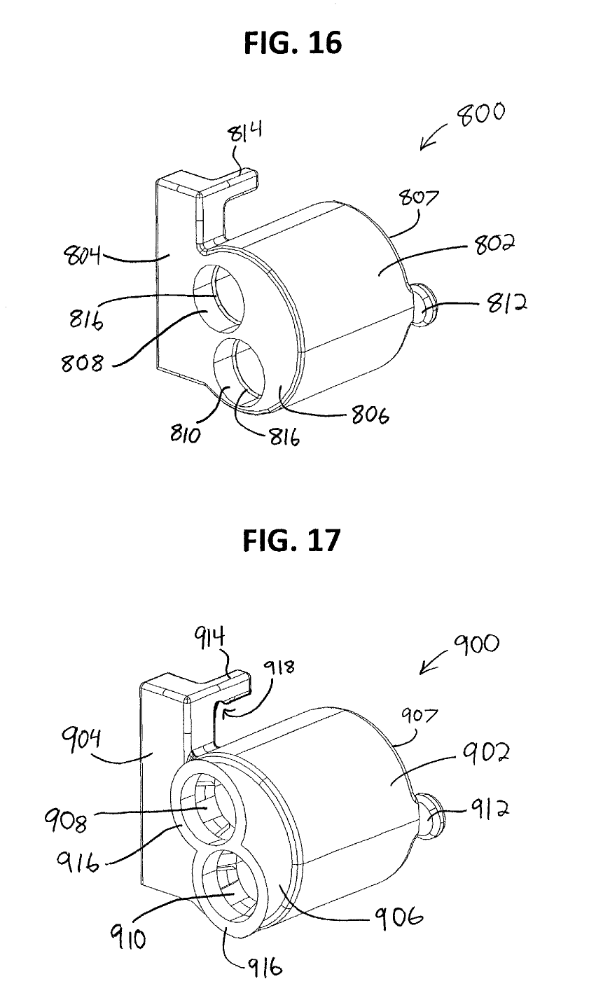

Referring to FIG. 16, guide device (800) comprises cylindrical portion (802) joined with rectangular portion (804). Guide device (800) also comprises front face (806) positioned proximally, rear face (807) positioned distally, and guide holes (808, 810), which extend through guide device (800) from front face (806) to rear face (807). Guide device (800) further comprises locking member (812), extending transversely relative to rear face (807), along cylindrical portion (802). Rectangular portion (804) includes arm (814), which extends in a rearward, or distal, direction from front face (806) toward rear face (807). Guide device (800) of this example further includes o-rings (816) that are positioned within guide holes (808, 810), as described in greater detail below.

Similarly, in FIG. 17, guide device (900) comprises cylindrical portion (902) joined with rectangular portion (904). Guide device (900) also comprises front face (906) positioned proximally, rear face (907) positioned distally, and guide holes (908, 910), which extend through guide device (900) from front face (906) to rear face (907). Guide device (900) further comprises locking member (912), extending transversely to rear face (907), along cylindrical portion (902). Rectangular portion (904) includes arm (914), which extends in a rearward, or distal, direction from front face (906) toward rear face (907). Rectangular portion (904) further includes a notch (918) in this example, located along an underside of arm (914). Guide device (900) of this example further includes inserts (916) that extend partially or fully within guide holes (908, 910), as described in greater detail below. As shown in FIG. 17, inserts (916) may be combined to form a single insert (916) that accommodates multiple guide holes (908, 910). Of course inserts (916) may be separate components as well, such as where each insert (916) is separate from the other insert (916) and accommodates a single corresponding guide hole (908, 910).

In some exemplary uses, guide devices (800, 900) may function similarly. Referring now to FIG. 19, guide device (800) may be prepared for insertion within an aperture (130a) of grid plate (96) by rotating guide device (800) about a longitudinal axis defined by guide device (800), such that locking member (812) is aligned with void space (820) of aperture (130a) of grid plate (896). In this orientation of guide device (800), arm (814) may be aligned with and inserted into another aperture (130b) located above aperture (130a). Of course, depending on the size of the apertures in grid plate (96), arm (814) may be aligned with an upper portion of the same aperture as locking member (812). Based on the teachings herein, it will be appreciated that various orientations of arm (814) may be suitable where arm (814) will not unduly interfere with the grid plate (96) during the locking steps discussed below.

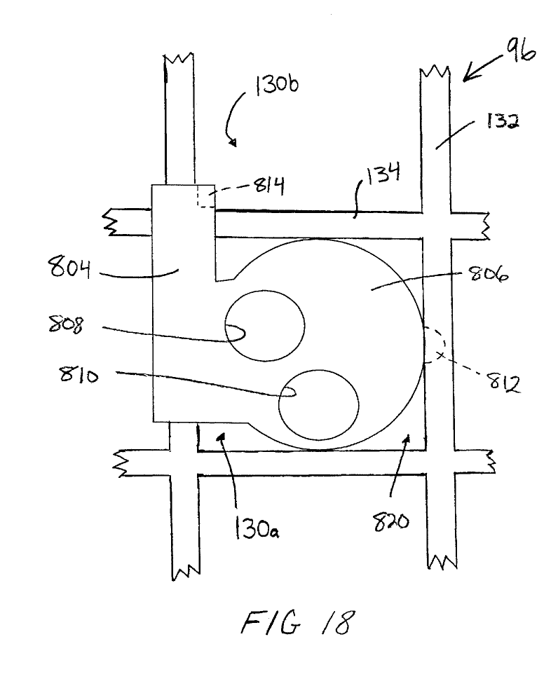

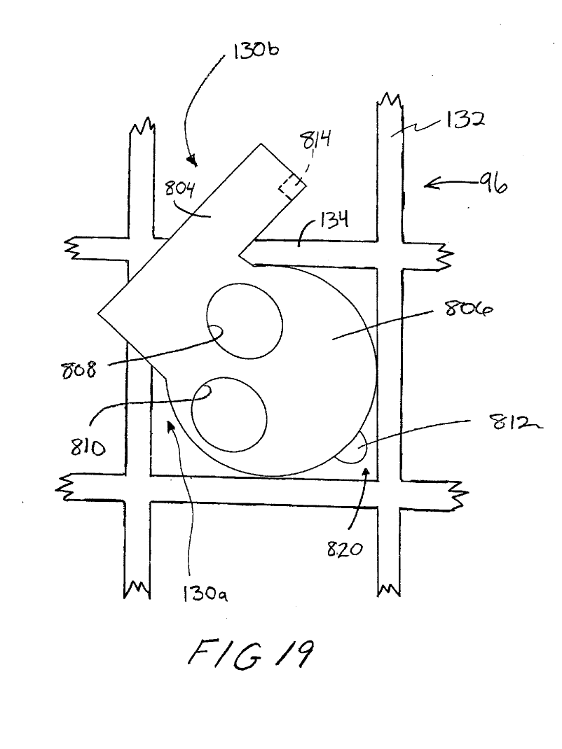

Once guide device (800) is oriented as shown in FIG. 10, guide device (800) may be inserted within aperture (130a) by moving guide device (800) distally. Once locking member (812) has extended beyond a rear side of grid plate (96), guide device (800) may be rotated counter-clockwise about a longitudinal axis approximately forty-five degrees into a locked position as shown in FIG. 18. In the locked position, locking member (812) is positioned behind the rear side of grid plate (96), thereby acting as a grounding structure to secure guide device (800) within aperture (130a). In other words, engagement between locking member (812) and the rear side of grid plate (96) may prevent proximal withdrawal of guide device (800) from aperture (130a) when guide device (800) has been rotated to the position shown in FIG. 18. Furthermore, arm (814) and rectangular portion (804) may abut a front side of grid plate (896) and interior wall of grid plate (896) respectively, thereby acting as a grounding structure to further secure guide device (800) within aperture (130a). In particular, rectangular portion (804) may prevent further distal insertion of guide device (800) into aperture (130a) of grid plate (96). Arm (814) may engage an adjacent vertical bar (132) of grid plate (96) and/or an adjacent horizontal bar (134) of grid plate (96), at or near a corner formed by adjacent bars (132, 134), to prevent further counter-clockwise rotation of guide device (800).

It should be understood that guide devices (800, 900) may also be configured to be compatible with grid plates that may include a backing substrate, e.g. backing substrate (136). In such versions where a backing substrate is present, in the locked position locking member (812) may be positioned distal of the backing substrate, thereby acting as a grounding structure to secure guide device (800, 900) within the aperture defined at least in part by the backing substrate. It should also be understood that some versions of grid plate (96) may have an internal slot or recess formed therein that is configured to accept rotational insertion of locking member (812) once guide device (800) has been inserted a sufficient distance into aperture (130a). Still other various types of grid plates (96) or other devices with which guide devices (800, 900) will be apparent to those of ordinary skill in the art in view of the teachings herein.

It should be understood that, in some versions, locking member (812) in the locked position shown in FIG. 18 prevents guide device (800) from proximal movement in use, while arm (814) and rectangular portion (804) prevent guide device (800) from distal movement in use as well as unintended further counter-clockwise rotation. It should be understood that, in some versions, locking member (812) and arm (814) with rectangular portion (804) may be operate to ground guide device (800) in the same or similar fashions as described above in the section relating to self-grounding guide cubes. In some other versions, guide device (800) may include some other locking feature that is movable relative to cylindrical portion (802). For instance, guide device (800) may include a resiliently-loaded locking member that is retracted when guide device (800) is initially inserted into aperture (130a), but that is triggered to deploy into a locking engagement with grid plate (96) when guide device (800) reaches a certain insertion depth in aperture (130a). Still other suitable features, components, or mechanisms for selectively locking and/or grounding guide device (800) relative to grid plate (96) will be apparent to those of ordinary skill in the art in view of the teachings herein.

It should further be understood that the locking and unlocking orientation of guide device (800) shown in FIGS. 18 and 19 is not intended to be limiting. In fact, other orientations may be used to secure guide device (800) within grid plate (896). By way of example only, before inserting guide device (800) into grid plate (96), guide device (800) may be rotated approximately ninety degrees in a clockwise direction with respect to the initial orientation shown in FIG. 19. In such an example, guide device (800) may be locked into position by rotating guide device (800) counter-clockwise by approximately forty-five degrees. Such an example would provide guide device (800) with a locked position having an orientation rotated approximately ninety degrees in a clockwise direction with respect to the locked orientation example shown in FIG. 18. Thus, it should also be understood that arm (814) may be positioned at any suitable corner defined by bars (132, 134) in aperture (130b) or at the corner of any other aperture (130) that is adjacent to or cater-cornered to aperture (130a). The various rotational orientations that guide device (800) may be positioned at for use may correspondingly allow guide holes (808, 810) to be selectively positioned within aperture (130a) of grid plate (896).

It should further be understood that the counter-clockwise rotation described above to lock guide device (800) to grid plate (96) (and clockwise rotation to unlock guide device (800) from grid plate (96)) is not intended to be limiting. In fact, other rotations may be used to move guide device (800) from a locked to unlocked state and vice versa. By way of example only, based on the teachings herein, it will be appreciated that in some configurations guide device (800) may be locked by rotating guide device (800) clockwise instead of counter-clockwise. Again, it should be understood that arm (814) may thus be positioned at any suitable corner defined by bars (132, 134) in aperture (130b) or at the corner of any other aperture (130) that is adjacent to or cater-cornered to aperture (130a).

Referring to FIGS. 16-19, locking members (812, 912) may have an arcuate shape as shown. It should be appreciated, however, that locking members (812, 912) may have any other suitable shape. For example, various thicknesses, lengths, widths, and other dimensions may be used for locking member (812, 912). Furthermore, the precise orientation of locking members (812, 912) is not intended to be limiting. For example, in some other versions, locking members (812, 912) may be positioned along rectangular portions (804, 904) respectively, while arms (814, 914) may be positioned along cylindrical portions (802, 902) respectively. Based on the teachings herein, other suitable orientations for locking members (812, 912) and arms (814, 914) will be appreciated by those of ordinary skill in the art. Furthermore, while guide devices (800, 900) are shown as being generally cylindraceous, it should be understood that guide devices (800, 900) may have any other suitable configuration.

In some versions guide devices (800, 900) may be compatible with grid plates having various thicknesses and aperture sizes. By way of example, in some versions, the length of cylindrical portions (802, 902) of guide devices (800, 900) may be of various sizes to accommodate various sized grid plates. In still other versions, the length of arms (814, 914) and/or other dimensions associated with arms (814, 914) may be of various sizes to accommodate various sized grid plates. Still yet, some versions may incorporate telescopic features to cylindrical portions (802, 902), rectangular portions (804, 904), and/or arms (814, 914) to accommodate various sized grid plates. Various other ways in which guide devices (800, 900) may be configured to accommodate various sized grid plates will be apparent to those of ordinary skill in the art in view of the teachings herein.

Referring now to FIG. 17, in some versions, guide device (900) may include notch (918) along an underside of arm (914). In some versions, notch (918) may be configured to work with some grid plates such that notch (918) latches onto a portion of the grid plate to provide for additional securing of guide device (900) with the grid plate. In still some other versions, notch (918) may be operatively configured to permit arm (914) to flex, and thereby permit arm (914) to deflect and clip onto a portion of the grid plate. For instance, arm (914) may deflect over a ridge presented by a grid plate as guide device (900) is being inserted into the grid plate; and arm (914) may resiliently snap or otherwise move back to a substantially straight orientation after notch (918) has been positioned over the ridge. Such engagement may provide a removable snap fit between guide device (900) and the grid plate. Of course, notch (918) may be entirely omitted from guide device (900) as well.

Guide cubes or devices (104, 104a, 104b, 104c, 800, 900), or components thereof, described herein may be made from rigid, pliable, and/or compressible materials. For example, suitable materials may include plastics, elastomers, ceramics, non-magnetic metals, among others. Guide cubes or devices (104, 104a, 104b, 104c, 800, 900), or components thereof, described herein may be made using a molding process, an extrusion process, or any other suitable manufacturing process. By way of example only, and not limitation, other suitable manufacturing processes may provide that some components may be stamped from non-magnetic metals. Furthermore, the guide devices may be constructed as separate components joined together or as single molded components. Where components are molded, single material constructions may be used or multiple material constructions may be achieved by over-molding or multi-step-molding processes. Various other suitable materials of construction and manufacturing processes to make guide cubes or devices (104, 104a, 104b, 104c, 800, 900) will be apparent to those of ordinary skill in the art in view of the teachings herein.

### D. Elastomeric Inserts

Referring again to FIG. 17, guide device (900) of the present example includes inserts (916). Inserts (916) may be operatively configured to assist in securing an instrument such as a biopsy device (14) or a portion of a biopsy device (14) (e.g., needle (90) of biopsy device (14), a combination of cannula (94) and obturator (92), etc.) within a selected guide hole (908, 910). Inserts (916) of this example fit within guide holes (908, 910), and may be comprised of an elastomeric material in some versions. Inserts (916) are designed such that the opening in the inserts (916) is smaller in diameter than the diameter of the instrument, e.g. cannula (94). When cannula (94) is inserted in guide hole (908, 910), insert (916) compresses to provide for a secure fit. In other words, while insert (916) permits insertion of cannula (94) or needle (90), etc., through a selected guide hole (908, 910), friction between the inserted instrument and the elastomeric material of insert (916) provides some resistance to axial movement of the inserted instrument relative to guide hole (908, 910). In some versions, the securing force provided by insert (916) is such that the compressed tissue of a patient will not displace cannula (94) proximally from guide hole (908, 910) during a biopsy procedure.

Based on the teachings herein, those of ordinary skill in the art will appreciate that inserts (916) may be removable and available in different sizes and with different compressive characteristics and/or frictional characteristics. Thus, in some versions, use of removable inserts (916) permits guide device (900) to be used with instruments of various diameters, such as various sized biopsy devices or various sized portions of biopsy devices, such as needles, cannulas, obturators, or combinations of these and other components.

In some versions inserts (916) may be integrally molded with guide device (900). In some such versions, inserts (916) may extend partially into guide holes (908, 910) or inserts (916) may extend fully through guide holes (908, 910). In some other versions, inserts (916) and guide device (900) are molded separately, and insert (916) is then secured to guide device (900) in any suitable fashion (e.g., using adhesive, etc.). Various suitable ways in which inserts (916) and guide device (916) may be molded together or otherwise secured together will be apparent to those of ordinary skill in the art in view of the teachings herein.

Inserts (916) may be constructed from any suitable compressive material or other type of material (e.g., any suitable material to provide friction). Based on the teachings herein, those of ordinary skill in the art will appreciate that several elastomeric materials may be suitable for use with inserts (916). By way of example only, suitable elastomeric materials may include thermosetting plastics that may require vulcanization, thermoplastic elastomers (e.g. Santoprene™ among others), natural rubber, synthetic rubbers (e.g. ethylene propylene diene M-class—EPDM—among others), and other polymers having suitable elastic properties.

### E. Retaining Rings

Referring now to FIG. 16, guide device (800) may include retaining rings (816) within guide holes (808, 810). Retaining rings (816) may be operatively configured to assist in securing an instrument such as a biopsy device (14) or a portion of a biopsy device (14) (e.g., needle (90) of biopsy device (14), a combination of cannula (94) and obturator (92), etc.) within a selected guide hole (808, 810). Retaining rings (816) may fit within or otherwise extend radially inwardly in guide holes (808, 810), and may be comprised of a compressible or elastomeric material in some versions. Retaining rings (816) of the present example are designed such that the inner diameter of the o-rings (816) is smaller than the outer diameter of the instrument, e.g. cannula (94). For instance, retaining ring (816) may define a relatively small inner diameter that is radially consistent throughout the circumference of the opening defined by retaining ring (816). Alternatively, retaining ring (816) may include several protrusions that project radially inward, with such protrusions collectively defining the relatively small inner diameter.

In some versions, when cannula (94) is inserted in a selected guide hole (808, 810), retaining ring (816) compresses to provide for a secure fit. In other words, while retaining ring (816) permits insertion of cannula (94) or needle (90), etc., through a selected guide hole (808, 810), friction between the inserted instrument and the elastomeric material of retaining ring (816) provides some resistance to axial movement of the inserted instrument relative to guide hole (816). In some versions, the securing force provided by retaining ring (816) is such that the compressed tissue of a patient will not displace cannula (94) proximally from guide hole (808, 810) during a biopsy procedure.

Based on the teachings herein, those of ordinary skill in the art will appreciate that retaining rings (816) may be removable and available in different sizes and with different compressive characteristics. Thus, in some versions, use of removable retaining rings (816) permits guide device (800) to be used with instruments of various diameters, such as various sized biopsy devices or various sized portions of biopsy devices, such as needles, cannulas, obturators, or combinations of these and other components. In some versions, retaining rings (816) may of various thickness such that the surface area contact between retaining rings (816) and guide holes (808, 810), and/or the surface area contact between retaining rings (816) and the instrument, may be increased or decreased as needed or desired

In some versions retaining rings (816) may be integrally molded with guide device (800). In some versions, retaining ring (816) is formed by a sheet that is molded within or otherwise provided within guide cube (800). Such a sheet may extend to reach both guide holes (808, 810). Thus, retaining ring (816) need not be truly ring-shaped. In some other versions, each retaining ring (816) is formed separately. By way of example only, retaining rings (816) may comprise conventional o-rings. Retaining rings (816) may thus be an accessory added to guide holes (808, 810) subsequent to fabricating guide device (800). For instance, an annular groove may be formed within the passageway of guide hole (808, 810), and an elastomeric member (e.g., rubber o-ring, etc.) may be positioned within the annular groove to act as a retaining ring (816). Such an elastomeric member may have an uncompressed outer diameter that is greater than the inner diameter defined by the passageway of guide hole (808, 810), and perhaps even greater than the inner diameter defined by the annular groove that is within the passageway of guide hole (808, 810). In some such versions, the elastomeric member may be positioned about cannula (94) before cannula (94) is inserted in guide hole (808, 810). As cannula (94) is inserted in guide hole (808, 810), the elastomeric member may compress to fit within guide hole (808, 810). When the elastomeric member reaches the annular groove, the elastomeric member may expand and fit within the annular groove. Still other ways in which a retaining ring (816) may be formed and/or provided within guide device (800) will be apparent to those of ordinary skill in the art in view of the teachings herein.

Retaining rings (816) may be constructed from any suitable material having any desired compressive and/or frictional characteristics. Based on the teachings herein, those of ordinary skill in the art will appreciate that several elastomeric materials may be suitable for use with retaining rings (816). By way of example only, suitable elastomeric materials may include thermosetting plastics that may require vulcanization, thermoplastic elastomers (e.g. Santoprene™ among others), natural rubber, synthetic rubbers (e.g. ethylene propylene diene M-class—EPDM—among others), and other polymers having suitable elastic properties. Alternatively, any other suitable material or combination of materials may be used to form retaining rings (816).

It should also be understood that each guide hole (808, 810) may have more than one associated retaining ring (816). For instance, each guide hole (808, 810) may have two or more retaining rings (810) that are axially staggered along the length of guide hole (808, 810). Furthermore, a guide device (800, 900) may have inserts (916) in addition to having retaining rings (810), among other various components.

As noted above, any guide cube or device (104, 104a, 104b, 104c, 800, 900) described herein may be used in a procedure that includes the use of PEM imaging, BSGI imaging, or any other suitable type of imaging. By way of example only, a guide cube or device (104, 104a, 104b, 104c, 800, 900) may be used with a grid plate (96) that is configured for use in an MRI setting, a grid plate for use in a nuclear/molecular imaging setting, or with some other type of cube holder (e.g., “guide holder”) used in nuclear/molecular imaging or other type of imaging. For instance, a suitable alternative cube holder or “guide holder” may include fewer openings (e.g., one to four) that are configured to receive a guide cube or device (104, 104a, 104b, 104c, 800, 900) as compared to the number of recesses (130) in grid plate (96). Furthermore, a guide cube or device (104, 104a, 104b, 104c, 800, 900) may be used with a biopsy device (14) in conjunction with a full targeting set or with just a biopsy device (14) (e.g., in settings where a radioisotope can be communicated through the biopsy device (14)). It should also be understood that a guide cube or device (104, 104a, 104b, 104c, 800, 900) may be used just with a radioisotope, without necessarily involving any biopsy device (14). For instance, a radioisotope may be provided on or through an implement that has a sharp tip, and the implement may be inserted through the guide cube or device (104, 104a, 104b, 104c, 800, 900). Still other various settings and combinations in which a guide cube or device (104, 104a, 104b, 104c, 800, 900) may be used will be apparent to those of ordinary skill in the art in view of the teachings herein.

While several guide devices, including guide cubes, have been discussed in detail above, it should be understood that the components, features, configurations, and methods of using the guide devices discussed are not limited to the contexts provided above. In particular, components, features, configurations, and methods of use described in the context of one of the guide devices may be incorporated into any of the other guide devices. One merely exemplary additional feature that may be provided in any of the guide devices described herein is one or more ridges on one or more external faces of the guide device. Such ridges may be substantially rigid, elastomeric, or have any other suitable properties. Such ridges may provide a more secure fit between a guide device and grid (e.g., reducing the likelihood that that the guide device will undesirably fall out of the grid plate), may permit a single guide device to be inserted in different grids having differently sized openings, and/or may provide other results. Still other additional and alternative suitable components, features, configurations, and methods of using the guide devices will be apparent to those of ordinary skill in the art in view of the teachings herein.

Versions of the present invention have application in conventional endoscopic and open surgical instrumentation as well as application in robotic-assisted surgery.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A guide device for guiding a medical instrument relative to a patient, the guide device being usable with a first plate and a second plate, wherein the first plate has a plurality of apertures, wherein the second plate and the first plate are adjustable to secure a portion of the patient, wherein the guide device is configured to be coupled with a selected one of the apertures of the first plate, the guide device comprising:
a. a first portion, wherein the first portion comprises:
i. at least one passageway, wherein the at least one passageway extends through the first portion, wherein the at least one passageway is configured to receive at least a portion of the medical instrument, and
ii. a locking member, wherein the locking member is operatively configured to contact a distal portion of the first plate, wherein the locking member is further operatively configured to prevent the guide device from proximal movement when the guide device is distally inserted within the selected one of the apertures of the first plate; and
b. a second portion, wherein the second portion is operatively configured to contact a proximal portion of the first plate, wherein the second portion is further operatively configured to prevent the guide device from further distal movement when the guide device is distally inserted within the selected one of the apertures of the first plate.

2. The guide device of claim 1, further comprising a protruding member, wherein the protruding member extends from the second portion in a distal direction, wherein the protruding member is operatively configured to contact a portion of an interior wall of the first plate, wherein the protruding member is further operatively configured to prevent the guide device from rotational movement when the guide device is inserted within the selected one of the apertures of the first plate.

3. The guide device of claim 2, wherein the protruding member contacts the portion of the interior wall of the first plate when the guide device is in a locked position.

4. The guide device of claim 3, wherein the portion of the interior wall of the first plate contacted by the protruding member is separate from an interior wall of the selected one of the apertures of the first plate.

5. The guide device of claim 2, wherein the protruding member of the second portion comprises a notch, wherein the notch is operatively configured to receive a portion of the first plate to further secure guide device when the guide device is inserted within the selected one of the apertures of the first plate.

6. The guide device of claim 1, wherein the guide device is operatively configured to move from an unlocked position to a locked position when the guide device is inserted within the selected one of the apertures.

7. The guide device of claim 6, wherein the guide device is insertable in the first plate along a direction of insertion, wherein the guide device is rotatable about a rotational axis to move the guide device from the unlocked position to the locked position, wherein the rotational axis is parallel to the direction of insertion.

8. The guide device of claim 7, wherein the guide device is rotated about forty-five degrees about the rotational axis to move the guide device from the unlocked position to the locked position.

9. The guide device of claim 6, wherein in the locked position the locking member contacts the distal portion of the first plate, wherein in the locked position the second portion contact the proximal portion of the first plate.

10. The guide device of claim 6, wherein in the unlocked position the locking member is positioned within a void space of the selected aperture of the first plate.

11. The guide device of claim 1, wherein the guide device is rotatable to position the at least one passageway to a selected orientation.

12. The guide device of claim 1, wherein the first portion forms a generally cylindrical shape.

13. The guide device of claim 1, wherein the second portion forms a generally rectangular shape.

14. The guide device of claim 1, wherein the locking member forms a generally arcuate shape.

15. The guide device of claim 1, wherein the guide device comprises two or more passageways, wherein the two or more passageways are generally parallel to each other.

16. A guide device for guiding a medical instrument relative to a patient, the guide device being insertable within a selected aperture of a grid plate, the guide device being operatively configured to resist proximal and distal movement when inserted within the selected aperture of the grid plate, the guide device comprising:
a. a first portion having a proximal face and distal face, wherein the first portion comprises one or more guide holes extending through the first portion from the proximal face to the distal face;
b. a second portion associated with the proximal face of the first portion;
c. a locking member associated with the distal face of the first portion, wherein the locking member protrudes laterally from the first portion; and
d. a protruding member associated with the second portion, wherein the protruding member extends distally from the second portion.

17. The guide device of claim 16, wherein the guide device is constructed from a single molded piece.

18. The guide device of claim 16, wherein the guide device further comprises one or more retaining rings positioned within the one or more guide holes, wherein the retaining rings are operatively configured to resist proximal movement of an inserted portion of the medical instrument.

19. The guide device of claim 16, wherein the guide device further comprises one or more elastomeric inserts within the one or more guide holes, wherein the inserts are operatively configured to resist proximal movement of an inserted portion of the medical instrument.

20. A method of securing a guide device within a selected aperture of a grid plate comprising, wherein the guide device comprises one or more guide holes extending through the guide device from a proximal face to a distal face, a proximal grounding structure, and a distal grounding structure, the method comprising:
a. aligning the guide device, wherein the act of aligning the guide device comprises:
i. positioning the guide device proximal to the selected aperture of the grid plate, and
ii. rotating the guide device to align the distal grounding structure with a void space of the selected aperture of the grid plate;
b. inserting the guide device within the selected aperture of the grid plate, wherein the distal grounding structure is positioned distal to the grid plate upon insertion of the guide device within the selected aperture of the grid plate; and
d. rotating the inserted guide device within the selected aperture of the grid plate to position the distal grounding structure and the proximal grounding structure in generally overlapping orientations with the grid plate.
